(19) United States
(12) Patent Application Publication
(10) Pub. No.: US 2024/0308953 A1
(43) Pub. Date: Sep. 19, 2024

(54) SOLUBLE CHELATOR FOR TARGETING RECOMBINANT PROTEINS

(71) Applicant: CUBE BIOTECH GMBH, Mannheim (DE)

(72) Inventors: Roland Fabis, Leverkusen (DE); Jan Kubicek, Cologne (DE); Maertens Barbara, Cologne (DE)

(21) Appl. No.: 18/263,637

(22) PCT Filed: Jan. 24, 2022
(86) PCT No.: PCT/EP2022/051492

§ 371 Date: Jul. 31, 2023

Related U.S. Application Data
(60) Provisional application No. 63/144,907, filed on Feb. 2, 2021.

(51) Int. Cl. C07C 229/76; C07C 229/26; C07C 237/10; G01N 33/20

(52) U.S. Cl. CPC C07C 229/76; C07C 229/26; C07C 237/10; G01N 33/20

(57) ABSTRACT

Soluble chelator-compounds, methods for their production and their use for the modification and/or immobilization of target molecules.

### FIELD OF THE INVENTION

The present invention relates to soluble chelator compounds, methods for their production and their use for the modification and/or immobilization of target molecules.

### BACKGROUND OF THE INVENTION

Chelation is a type of bonding of ions and molecules to metal ions. It involves the formation or presence of two or more separate coordinate bonds between a polydentate (multiple bonded) ligand and a single central atom.

These ligands are called chelants, chelators, chelating agents, or sequestering agents.

Chelation is useful in applications such as providing nutritional supplements, in chelation therapy to remove toxic metals from the body, as contrast agents in MRI scanning, in manufacturing using homogeneous catalysts, in chemical water treatment to assist in the removal of metals, and in fertilizers.

In recent years, chelators were also developed for use in protein purification and manipulation.

For example, a polyhistidine-tag, that is an amino acid motif in proteins that typically consists of at least six histidine (His) residues, is often attached to the N- or C-terminus of a protein. This His-tag is also known as “hexa histidine-tag, 6×His-tag, His-6 tag”. The polyhistidine-tags is able to bind to metal ions and, thus, enables the binding of chelator to the tag. As such immobilized chelator may be used for affinity purification of genetically modified proteins and soluble chelator may be used for staining, certain purification methods, complex-building and other purposes.

However, prior art chelators, for example, such which rely on modified nitrilotriacetic acid (NTA), do not possess enough affinity in order to be used for a range of protein applications. This is on one hand because due to the rather weak binding the bond is lost is under stress, e.g. during applications with many washing steps and/or during gel electrophoresis. Furthermore, during protein production chelators are used which compete for binding with the chelator which should facilitate the desired function, such as for example immobilization or dye.

For example, tetradentate chelators, such as NTA, lose metal ions during purification, when other chelator, such as EDTA, or reductants, such as DTT, are present in the lysis buffer. EDTA is a very strong chelator, which removes membrane-stabilizing magnesium ions and is necessary for inhibiting metalloproteases in lysis buffer.

However, it removes almost all the metal ions from NTA resin if its concentration is high. Addition of DTT (dithiothreitol), which helps stabilizing the protein SH groups against oxidation to disulfides and protein dimerization, reduces metal ions, such as nickel, to the uncharged metal, so that the protein binding affinity is significantly lowered.

Thus, a great need exists in the field for stronger chelator, which can compete more effectively with the chelator already present during protein production.

### SUMMARY OF THE INVENTION

The present invention presents soluble chelators, which are strong enough to compete with chelators, such as EDTA or DTT, which are present during many protein production methods.

In fact, the chelators of the present invention bind so strongly to metal ions, such as for example Ni, so that solutions of EDTA and/or DTT with a concentration, which are usual in the preparation and purification of recombinant proteins, do not dissolve or reduce the ion, as NTA does.

The soluble chelators of the present invention can be used for selective staining and labeling of his-tagged proteins in gels and on chromatography columns, as well as for selective functionalization of a his-tagged protein in solution by a dye, a nanoparticle, a tool for click chemistry, a radioactive molecule, or an enzymatic function.

Furthermore, the soluble chelators of the invention can also be used for expression control in preparation of a recombinant protein and for quantitative determination of proteins. In particular, the soluble chelators of the present invention show very high affinity, strong, tight association and extremely slow dissociation, compared to the standard Ni-NTA, described in the prior art (e.g. EP0253303), and Tris NTA (e.g. described in WO 2006/013042), and allows its use as an alternative to antibodies in Western Blot or in affinity studies, e.g. by surface plasmon resonance.

In one aspect, the invention pertains to a soluble chelator comprising at least one amino-carboxylic acid of the general structure as depicted in formula I:

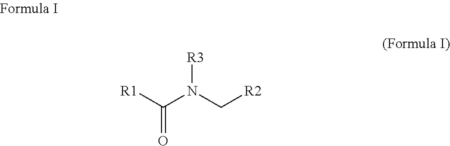

wherein R1 is selected from a chelating carboxylic acid or a chelator-group, such as formula II:

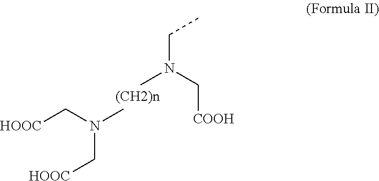

wherein n=0, 1, 2, or 3;
or R1 is selected from formula III:

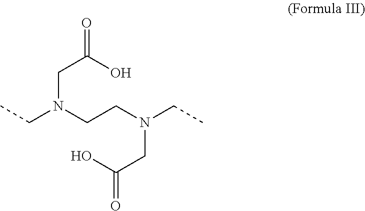

wherein each of the sides can be bound to R1;
and wherein
either
two molecules of formula I are bound to each other bivalently via R2 resulting in a core-molecule of formula IV:

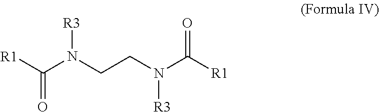

or
wherein three molecules of formula I are bound trivalent via R2 to formula V:

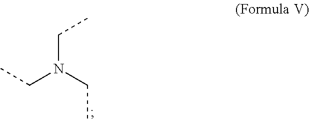

and
wherein R3 of the formulas mentioned above is selected either from the group comprising H, COOH, CH2-COOH and another molecule of formula I which is bound via R3-(CH₂)ₙ—R2, n=0, 1, 2, or 3.

According to a definition of the IUPAC,” chelator” means a polydentate ligand, which can be involved in formation of at least two coordinative bonds. In this invention the chelator are aminopolycarboxylic acids, such as EDTA (ethylenediamine tetraacetic acid), DTPA (diethylene triamine pentaacetic acid), TTHA (triethylenetetramine hexaacetic acid), EDDS (Ethylenediamine-N,N′-disuccinic acid), DOTA (1,4,7,10-tetraazacyclododecane-1,4,7,10-tetraacetic acid), NOTA (1,4,7-triazacyclononane-triacetic acid), and the like.

In the present invention, the carboxylic acid groups are written in the acid form, but with a pH between 6 and 8.5, which is common for the affinity purification of recombinant proteins, the biggest part of the carboxylic acid groups is in the carboxylate form, and some amino groups are protonated. In order to keep the description simple and clear, in the text and claims these groups are written as “COOH” and NH₂.

The chelator could contain other functional groups, such as carboxylic acids, alcohol, thiol, and the like as long as they do not interfere the functionality.

Examples for difunctional linkers are ethylene diamine, propylene diamine, diamino polyethylene glycol, and the like.

Examples for trifunctional linkers are 1,1,1-tris-(aminomethyl)-ethane, tris-(3-aminopropyl)-amine, tris-(2-aminoethyl)-amine, diethylenetriamine, 3-(2-aminoethyl)-pentane-1,5-diamine, and amino-functionalized trifunctional branched PEGs, basing on a glycerol core.

One possibility for the synthesis of the branched and linker-connected chelator is basing on an activated chelator, which can react with the amino groups of the linkers, so, e.g. EDTA monoanhydride or dianhydride. Alternatively, chelator and linker can be mixed in the ratio, which reflects the ratio in the resulting product, and coupling can be started by adding one or more condensing agents, such as carbodiimides, like EDC or DCC, or other reagents used in peptide syntheses.

After synthesis and coupling of the chelator chains, the reaction product can be charged with metal ions, which can bind the desired target proteins. As Ni, Cu, Co, Zn, Fe, Eu, Sc are suitable for reversible protein binding, nickel and cobalt are preferred for purification of His-tagged proteins, while iron and alumina are preferred for the isolation of phosphoproteins. For instance, the affinity of the chelator to the metal follows the sequence Cu>Ni>Zn>Co.

Thus, in another aspect, the soluble chelator comprises a positively charged metal-ion selected from the metals Ni, Co, Cu, Fe, Ca, Zn, Al, Eu, Ga, and Sc. Such positively charged metal-ions are for example: Ni⁺, Ni²⁺, Ni³⁺, Ni⁴⁺, Co²⁺, Cu²⁺, Cu³⁺, Fe²⁺, Fe³⁺, Ca²⁺, Zn²⁺, Pb²⁺, Al³⁺, Eu³⁺, Ga³⁺, Sc³⁺, Cr³⁺, Zr⁴⁺.

In another aspect, the soluble chelator comprises a number of carboxy groups of at least 8, at least 10, at least 12 and up to 14, up to 16.

In yet another aspect, the soluble chelator comprises a number of amino groups of at least 6, at least 8, at least 10, at least 12 and up to 14, up to 16.

In yet another aspect, the soluble chelator comprises R1 which is selected from the group comprising EDTA (ethylenediamine tetra-acetic acid), EGTA (ethylene glycol-bis-(β-aminoethyl ether)-N,N,N′,N′-tetra-acetic acid), DTPA (diethylenetriamine penta-acetic acid), and EDDS (ethylenediamine-N,N′-di-succinic acid).

In yet another aspect, the soluble chelator is depicted in formula VI:

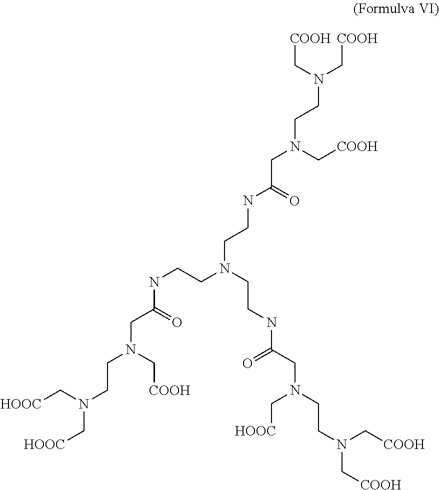

In yet another aspect, the soluble chelator is depicted in formula VII:

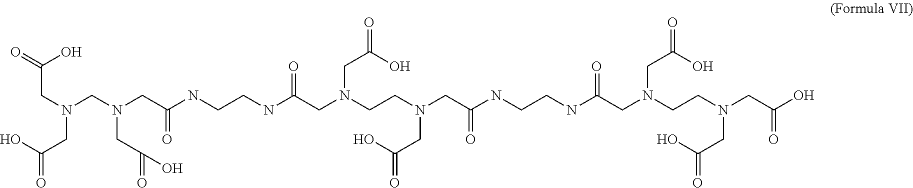

In yet another aspect, the soluble chelator is depicted in formula VIII:

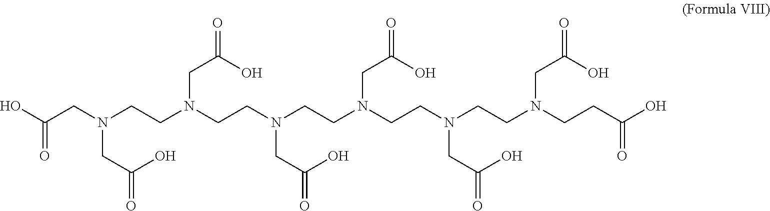 [SMILES: O=C(O)CCN(CCN(CCN(CCN(CCN(CCN(CC(=O)O)CC(=O)O)CC(=O)O)CC(=O)O)CC(=O)O)CC(=O)O)CC(=O)O]

The chelators of formulae I to VIII can be functionalized by a number of modifications as described hereinunder.

In yet another aspect, the soluble chelator is further modified to comprise a tag, such as a biotin, desthiobiotin, a His-tag, a strep-tag, a FLAG-tag, a Rho-tag (e.g. Rho-1D4-tag).

In yet another aspect, the soluble chelator is further modified to comprise a dye, a colorant, a fluorophor, a nanoparticle, a functional group for click chemistry, a radioactive molecule, or a molecule with enzymatic activity. Per molecule one, two, or three molecules can be coupled. The chelator groups with the molecule attached are then reduced to a tetradental form.

In yet another aspect, the present invention also pertains to methods for producing the soluble chelator as mentioned herein, comprising the steps of:
a) Providing EDTA dianhydride and a trifunctional linker in a ratio of 4:1 to 2:1, preferably 2.75:1 to 3.25 to 1;
b) Coupling said dianhydride to said trifunctional linker via amine group onto the linker, wherein the linker reacts with three different dianhydrides, so that a substance (APA1)3-L is formed, wherein APA1 is EDTA with one anhydride group and one amide function, and L is the trifunctional linker;
c) Providing a molecule, which can be a dye, a colorant, a Fluorophor, a nanoparticle, a functional group for click chemistry, a radioactive molecule, or a molecule with enzyme activity, comprising one or more amine groups;
d) Coupling said linker-connected dianhydride to said molecule via amine group, wherein a single, two, or three carboxy groups per aminocarboxylic acid reacts with the amine moiety;
e) Providing water or an aqueous puffer to hydrolyze the remaining anhydride groups;
f) Immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

In yet another aspect, the present invention also pertains to methods for producing the soluble chelator as mentioned herein, comprising the steps of:
a) Providing EDTA monoanhydride and a trifunctional linker in a ratio of 4:1 to 2:1, preferably 2.75:1 to 3.25 to 1;
b) Coupling said monoanhydride to said trifunctional linker via amine group onto the linker, wherein the linker reacts with three different monoanhydrides, so that a substance (APA2)3-L is formed, wherein APA2 is EDTA with one amide function, and L is the trifunctional linker;
c) Providing a molecule, which can be a dye, a colorant, a Fluorophor, a nanoparticle, a functional group for click chemistry, a radioactive molecule, or a molecule with enzyme activity, comprising one or more amine groups;
d) Coupling said linker-connected EDTA to said molecule via amine group, wherein a single, two, or three carboxy groups per aminocarboxylic acid reacts with the amine moiety, by means of one or more condensing agents, such as carbodiimides, like EDC or DCC, or other reagents used in peptide syntheses;
e) Immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

In yet another aspect, the present invention also pertains to methods for producing the soluble chelator as mentioned herein, comprising the steps of:
a) Providing a solid phase resin, which is able to bind carboxylic acid groups, such as Wang resin, and EDTA, with two carboxylic acid protective groups, such as methyl ester or tert butylester;
b) Coupling said EDTA derivative to said solid phase resin by means of one or more condensing agents, such as carbodiimides, like EDC or DCC, or other reagents used in peptide syntheses;
c) Providing a trifunctional linker;
d) Coupling said trifunctional linker onto the remaining carboxylic acid group of the solid-phase bound EDTA;
e) Removal of the protective groups by trifluoroacetic acid, or the like (such as other aqueous acids);
f) Cleavage of the aminopolycaroxylic acid from the solid phase by trifluoroacetic acid, or the like;
g) Providing a molecule, which can be a dye, a colorant, a Fluorophor, a nanoparticle, a functional group for click chemistry, a radioactive molecule, or a molecule with enzyme activity, comprising one or more amine groups;
h) Coupling said linker-connected dianhydride to said molecule via amine group, wherein a single, two, or three carboxy groups per aminocarboxylic acid reacts with the amine moiety;
i) Immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

In yet another aspect, the present invention also pertains to uses of the soluble chelator of the present invention as a colorant, a dye, a fluorophor, a nanoparticle, as a tool for click-chemistry, a radioactive molecule or as a molecule with enzymatic function.

In yet another aspect, the present invention also pertains to the use of the soluble chelator for binding to a target protein comprising a tag which binds to metal ions, such as for example a polyhistidine-tag.

Polyhistidine-tags are common in molecular biology and can be used for separating recombinant proteins, expressed in bacteria, such as E. coli, yeast, and mammalia. Thus, His-tagged proteins bind with metal ion-loaded chelator at pH values, where the histidine groups are non-protonated, while the great majority of the remaining proteins do not interact with the metal ions. When the pH value is lowered and histidine becomes protonated, the interaction of the protein to the metal ion is cleared, and the protein can be separated from the metal ion and/or the chelate-complex. Alternatively, the separation can also be performed with applying high concentrations of imidazole, which binds onto the metal ions and replaces the his-tags, which leads to be separation of the protein from the metal ion and/or the chelate-complex. For this application, imidazole concentrations of 100 to 300 mM are commonly used.

In yet another aspect, the present invention also pertains to the use of the soluble chelator for complex formation.

In yet another aspect, the present invention also pertains to the use of the soluble chelator for detection of metal ions, preferably in combination with a His-tag or biotinylation.

In yet another aspect, the present invention also pertains to the use of the soluble chelator for binding onto functionalized streptavidin.

The soluble chelator of the present invention shows significant higher metal binding affinity as compared to conventional tetradentate chelators. For example, the metal binding capacity, determined with Cu²⁺, shows at least 750-2.000 μmol/mg, more preferred at least 800-1.500 μmol/mg. In addition to that, extremely high quantities of His-tagged proteins can be bound with the soluble chelator according to this invention. So, in the following examples up to 1 mmol protein per mg chelator can be bound by the soluble chelator of the invention.

The affinity of the soluble chelator of the present invention for His-tagged proteins is much higher, compared to the NTA analoga. So, immobilized for surface plasmon resonance measurements, there is no visible protein loss from the chip, while NTA and variants are showing protein leaching during wash. So, a stable, non-reversible protein binding via affinity bonds between metal and poly-His is feasible.

Moreover, because of the multimeric nature of the chelator, they are able to bind His-tagged proteins with a cooperative effect, which gives a much higher affinity than a monomeric chelator alone.

Thus, due to the strong affinity binding, the soluble chelator according to this invention show an extremely good resistance against solution-based chelating agents and reductants, e.g. even when 5 mM Imidazol, 10 mM Imidazol, or even 20 mM Imidazol are used the soluble chelator according to this invention maintain their protein-binding capacity. The same applies to protein-solutions comprising, alone or additionally to Imidazol, at least 5 mM, at least 10 mM, or even at least 20 mM EDTA; and/or at least 5 mM, at least 10 mM, or even at least 20 mM DTT. So, applications with e.g. proteins from eucaryotic systems are possible, such as SDS gel, blots, pull-downs, labelling of surface proteins in an expression, which cannot be done with conventional materials such as tris-NTA.

Due to the high resistance against chelator and reductants the soluble chelator according to this invention are particularly suitable for the binding to membrane proteins, especially GPCRs. For the membrane protein binding in many cases detergents, such as dodecyl maltoside, or n-tetradecyl phospho-choline (FOS-14) are used to stabilize the protein in aqueous solution against precipitation and denaturation. The soluble chelator according to this invention show high protein binding capacity and a good tolerance against said detergents.

Although the soluble chelator according to this invention is very suitable for protein-binding, also single-stranded nucleic acids may be bound selectively. This method is based upon reversible binding of accessible imidazyl moieties, as present in single-stranded nucleic acids, to metal ions. Such accessible imidazyl moieties are not present in double-stranded nucleic acids and, thus, a selective binding of the soluble chelator according to this invention to single-stranded nucleic acids may be used for example to separate single-stranded from double-stranded nucleic acids, for example by complexing, staining, etc. In one embodiment, such a selective binding may be used in a half-quantitive assay in order to tell single-stranded viral RNA within a sample (e.g. a sputum-sample of a patient).

Of course, the soluble chelator according to this invention is very suitable for the removal of metal ions from solutions. EDTA forms metal complexes with stability constants of about 14 to 25 (Martell A. E. & Smith R. M. (1982), Critical Stability Constants, Vol. 5: First Supplement, Plenum Press, New York), so the soluble chelator according to this invention can efficiently be used to bind metal ions, such as Ni²⁺, Co²⁺, Mn²⁺, Cr³⁺, Pb²⁺, etc. This may be applied during assays testing water quality with respect to metal-ion contamination and similar purposes. The content of metal ions, especially toxic and/or radioactive metals, in a solution may also be reduced by complexing the metal ions with the chelator of the present invention within the liquid and applying a purification step via a tag-moiety attached to the chelator. One example is to attach the soluble chelator according to this invention to magnetic particles, which can be added to a solution, mixed and removed by a magnetic separation after binding to metal ions.

### DESCRIPTION OF EMBODIMENTS

### Examples

### Example 1: Synthesis and Functionalization of Tris-EDTA, Linked by Tris(2-Aminoethyl)Amine

### Synthesis of EDTA Monoanhydride

30.0 g (117 mmol) EDTA dianhydride were dissolved in 200 ml DMSO under N₂ atmosphere. Then a mixture of 2.1 ml (117 mmol) water in 10 ml DMSO is added dropwise, and the mixture was stirred for three hours while a precipitate has formed. The mixture was cooled, suction-filtered and washed repeatedly with dry DMSO and diethyl ether. The precipitate was dried and used for the next steps.

Reaction of EDTA Monoanhydride with Tris(2-Aminoethyl)-Amine

1 g (3.65 mmol) EDTA monoanhydride were dissolved in 30 ml DMSO under nitrogen, and a solution of ml tris(2-aminoethyl)-amine (mmol) in 10 ml DMSO was added dropwise. After the addition is finished, the suspension was stirred two hours at ambient temperature and three hours at 60° C. The reaction mixture was evacuated under high vacuum, and the remaining product could be used for the next steps.

Functionalization of the Product with Different Fluorophors and Nickel

5×100 mg (5×108 micromol) of the reaction product are dissolved each in 50 ml water. Then 5×25.0 mg of EDC (5×130 micromol), 5×15.0 mg of N-hydroxy succinimide (5×130 micromol), and 69.3 mg (110 micromol) CY3-amine (Lumiprobe GmbH, Hannover, Germany), 47.0 mg (110 micromol) BDP-FL amine (Lumiprobe), 56.2 mg (110 micromol) FAM amine, 5 isomer (Lumiprobe), 90.4 mg (110 micromol) Atto 633 amine (Atto-Tec GmbH, Siegen, Germany), and 79.2 mg (110 micromol) Alpha Fluor™ 488 amine (AAT Bioquest Inc, Sunnyvale, CA, USA) were added, and the reaction mixture was stirred two hours. Then 105 mg (40 micromol) nickel sulfate hexahydrate were added to each tube, and the reaction mixture was stirred another two hours. The product was dried under high vacuum, washed several times with dry diethyl ether, and dried again. In the next step the conjugates were purified over a 5 ml PureCube Q Cartridge (Cube Biotech GmbH) and eluted with a 0 ech Gmb NaCl gradient using an FPLC system.

### Example 2: Synthesis and Functionalization of a Linear EDTA-EDA-EDTA-EDA-EDTA Molecule

30.0 g (117 mmol) EDTA dianhydride were dissolved in 200 ml DMSO under N2 atmosphere. 5.21 ml (78 mmol) ethylene diamine were dissolved in 25 ml DMSO and added dropwise to the reaction mixture. After finishing the addition, the mixture was stirred two hours at ambient temperature, and three hours at 60° C. Then the reaction mixture was evacuated under high vacuum, and the remaining product could be used for the next steps.

Functionalization of the Product with Different Fluorophors and Nickel

5×100 mg (5×108 micromol) of the reaction product are dissolved each in 50 ml water. Then 5×25.0 mg of EDC (5×130 micromol), 5×15.0 mg of N-hydroxy succinimide (5×130 micromol), and 69.3 mg (110 micromol) CY3-amine (Lumiprobe GmbH, Hannover, Germany), 47.0 mg (110 micromol) BDP-FL amine (Lumiprobe), 56.2 mg (110 micromol) FAM amine, 5 isomer (Lumiprobe), 90.4 mg (110 micromol) Atto 633 amine (Atto-Tec GmbH, Siegen, Germany), and 79.2 mg (110 micromol) Alpha Fluor™ 488 amine (AAT Bioquest Inc, Sunnyvale, CA, USA) were added, and the reaction mixture was stirred two hours. Then 105 mg (40 micromol) nickel sulfate hexahydrate were added to each tube, and the reaction mixture was stirred another two hours. The product was dried under high vacuum, washed several times with dry diethyl ether, and dried again. In the next step the conjugates were purified over a 5 ml PureCube Q Cartridge (Cube Biotech GmbH) and eluted with a 0-2.5 M NaCl gradient using an FPLC system.

### Example 3: Solid Phase Synthesis and Functionalization of Tris-EDTA, Linked by Tris-(2-Ami-Noethyl)-Amine

Coupling of EDTA-Bis-Methylester onto Wang Resin

In a round-bottom flask 0.5 g Wang resin (Merck Millipore, Darmstadt, Germany) is covered with DMF and allowed to swell for 30 min. In another round-bottom flask 2 g EDTA-bis-methyl ester (preparation described in Govender, Journal of Membrane Science 279 (2006) 120-128) is dissolved in dry Dichloromethane under argon atmosphere. A small amount of DMF is added to achieve complete dissolution. The solution is cooled to 0° C. and 1 g N, N′-Diisopropylcarbodiimide is slowly added. The mixture is stirred for 20 min at the same temperature followed by evaporation. The residue is dissolved in 5 ml DMF and added to the resin suspension followed by addition of 100 mg 4-Dimethylaminopyridine. The suspension is shaken at room temperature for 1 h. For capping the rest of the free hydroxyl groups, 250 μl of acetic anhydride and 500 μl pyridine are added to the reaction mixture. The mixture is shaken at room temperature for additional 30 min. Resin is filtered, washed with DMF (3×), DCM (3×), MeOH (3×) and dried.

### Coupling of Tris-Aminoethyl-Amin

The functionalized Wang Resin is resuspended in 5 m DMF, and after 30 minutes 1 ml tris-(2-aminoethyl)-amine), 1.5 g 2-(1H-Benzotriazol-1-yl)-1,1,3,3-tetramethyluronium tetrafluoroborate (TBTU, Sigma Aldrich, Schnelldorf, Germany) and 2 g ethyl-diisopropylamine in 5 ml DMF are added. After 60 minutes incubation the product, the resin is suction-filtered and washed five times with DMF.

### Second Coupling of EDTA-Bis-Methyl Ester

The functionalized Wang Resin is resuspended in 5 m DMF, and after 30 minutes 1.5 g EDTA-bis-t-butyl ester, 1.5 g TBTU, and 3 ml ethyl-diisopropylamine in 5 ml DMF are added. After 60 minutes incubation the product, the resin is suction-filtered and washed five times with DMF. Then the product is washed by dichloromethane and methanol and dried under vacuum.

Cleavage from Solid Phase, Removal of Methyl Protective Groups

The Wang resin is transferred to a sintered glass funnel with fine porosity and washed three times with DMF, then five times with dichloromethane. The resin is resuspended in 50% TFA in DCM (v/v), and the mixture is stirred for three hours. Now the resin is filtered and washed three times with small portions of TFA. The filtrates are combined, and 100 ml cold diethyl ether is added to precipitate the peptide. The peptide is filtered through a fine sintered glass funnel, washed with cold ether, and dried.

1. A soluble chelator comprising at least one amino-carboxylic acid of the general structure as depicted in formula I: 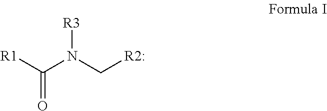
wherein R1 is selected from a chelating carboxylic acid or a chelator-group, such as formula II: 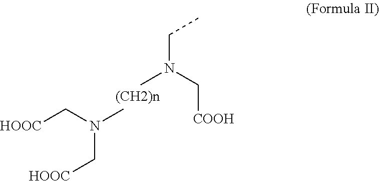
wherein n=0, 1, 2, or 3;
or R1 is selected from formula III: 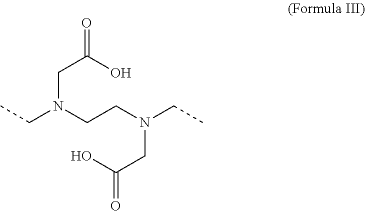
wherein each of the sides can be bound to R1; and
wherein
either
two molecules of formula I are bound to each other bivalently via R2 resulting in a core-molecule of formula IV: 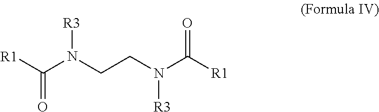
or
wherein three molecules of formula I are bound trivalent via R2 to formula V: 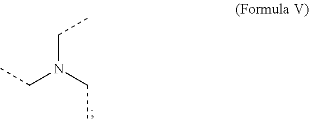
and
wherein R3 of the formulas above is selected either from the group comprising H, COOH, CH₂—COOH and another molecule of formula I which is bound via R3-(CH₂)ₙ—R2, n=0, 1, 2, or 3.

2. The soluble chelator of claim 1, further comprising a positively charged metal-ion selected from the metals Ni, Co, Cu, Fe, Ca, Zn, Al, Eu, Ga, and Sc.

3. The soluble chelator of claim 1, wherein the number of carboxy groups is at least 8.

4. The soluble chelator claim 1, wherein the number of amino groups is at least 6.

5. The soluble chelator of claim 1, wherein R1 is selected from the group comprising EDTA (ethylene diamine tetra acetic acid), EGTA (ethylene glycol-bis-(β-aminoethyl ether)-N,N,N′,N′-tetra acetic acid), DTPA (diethylene triamine penta acetic acid), and EDDS (ethylene diamine-N,N′-di succinic acid).

6. The soluble chelator of claim 1, as depicted in formula VI:

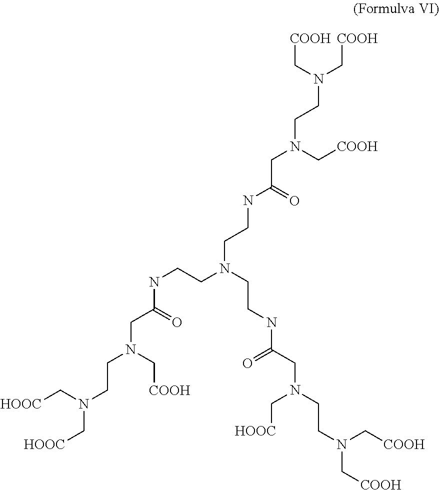

7. The soluble chelator of claim 1, as depicted in formula VII:

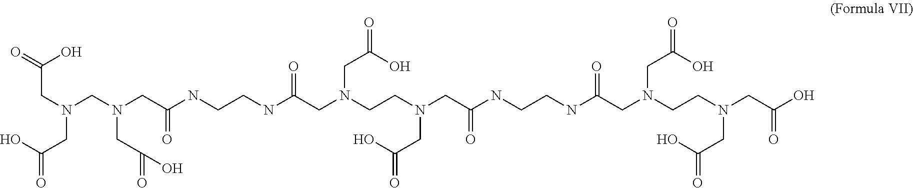

8. A soluble chelator as depicted in formula VIII:

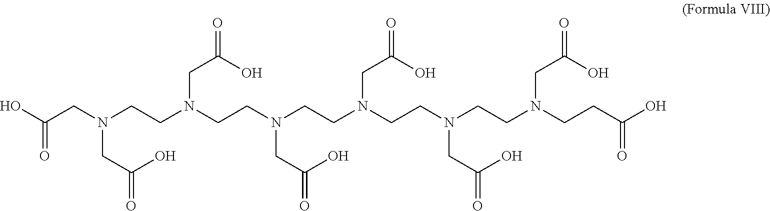 [SMILES: O=C(O)CCN(CCN(CCN(CCN(CCN(CCN(CC(=O)O)CC(=O)O)CC(=O)O)CC(=O)O)CC(=O)O)CC(=O)O)CC(=O)O]

9. The soluble chelator of claim 1, wherein the chelator is further modified to comprise a tag, such as a biotinylation, a His-tag, a strep-tag, a FLAG-tag, a Rho-tag (e.g. Rho-1D4-tag).

10. A method for producing the soluble, chelators of claim 1, comprising the steps of:
a. providing EDTA dianhydride and a trifunctional linker in a ratio of 4:1 to 2:1, preferably 2.75:1 to 3.25 to 1;
b. coupling said dianhydride to said trifunctional linker via amine group onto the linker, wherein the linker reacts with three different dianhydrides, so that a substance (APA1)3-L is formed, wherein APA1 is EDTA with one anhydride group and one amide function, and L is the trifunctional linker;
c. providing a molecule, which can be a dye, a colorant, a fluorophor, a nanoparticle, a functional group for click chemistry, a radioactive molecule, or a molecule with enzyme activity, comprising one or more amine groups;
d. coupling said linker-connected dianhydride to said molecule via amine group, wherein a single, two, or three carboxy groups per aminocarboxylic acid reacts with the amine moiety;
e. providing water or an aqueous puffer to hydrolyze the remaining anhydride groups; and
f. immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

11. A method for producing the soluble chelators of claim 1, comprising the steps of:
a. providing EDTA monoanhydride and a trifunctional linker in a ratio of 4:1 to 2:1, preferably 2.75:1 to 3.25 to 1;
b. coupling said monoanhydride to said trifunctional linker via amine group onto the linker, wherein the linker reacts with three different monoanhydrides, so that a substance (APA2)3-L is formed, wherein APA2 is EDTA with one amide function, and L is the trifunctional linker;
c. providing a molecule, which can be a dye, a colorant, a fluorophor, a nanoparticle, a functional group for click chemistry, a radioactive molecule, or a molecule with enzyme activity, comprising one or more amine groups;
d. coupling said linker-connected EDTA to said molecule via amine group, wherein a single, two, or three carboxy groups per aminocarboxylic acid reacts with the amine moiety, by means of one or more condensing agents, such as carbodiimides, like EDC or DCC, or other reagents used in peptide syntheses; and
e. immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

12. A method for producing the soluble chelators of claim 1, comprising the steps of:
a. providing a solid phase resin, which is able to bind carboxylic acid groups, such as Wang resin, and EDTA, with two carboxylic acid protective groups, such as methyl ester or tert butylester;
b. coupling said EDTA derivative to said solid phase resin by means of one or more condensing agents, such as carbodiimides, like EDC or DCC, or other reagents used in peptide syntheses;
c. providing a trifunctional linker;
d. coupling said trifunctional linker onto the remaining carboxylic acid group of the solid-phase bound EDTA;
e. removing the protective groups by trifluoroacetic acid, or the like;
f. cleaving the aminopolycaroxylic acid from the solid phase by trifluoroacetic acid, or the like;
g. providing a molecule, which can be a dye, a colorant, a fluorophor, a nanoparticle, a functional group for click chemistry, a radioactive molecule, or a molecule with enzyme activity, comprising one or more amine groups;
h. coupling said linker-connected dianhydride to said molecule via amine group, wherein a single, two, or three carboxy groups per aminocarboxylic acid reacts with the amine moiety;
i. immobilizing metal ions by contacting the solid phase-immobilized aminopolycarboxylic acid compound with solutions of metal ions, selected from Ni²⁺, Co²⁺, Cu²⁺, Zn²⁺, Al³⁺, Fe³⁺, Eu³⁺, Ga³⁺, Mn²⁺, Ca²⁺.

13. Use of the soluble, chelators of claim 1 as a colorant, a dye, a fluorophor, a nanoparticle, as a tool for click-chemistry, a radioactive molecule or as a molecule with enzymatic function.

14. Use of the soluble chelators of claim 1 for binding to a target protein comprising a tag which binds to metal ions, such as for example a polyhistidine-tag.

15. Use of the soluble, chelators of claim 1 for complex formation.

16. Use of the soluble chelators of claim 1 for detection of metal ions, preferably in combination with a His-tag or biotinylation.

17. Use of the soluble chelators of claim 1 for binding onto functionalized streptavidin.
